(19) United States
(12) Patent Application Publication
(10) Pub. No.: US 2004/0161470 A1
(43) Pub. Date: Aug. 19, 2004

(54) Preparation of polyphosphazene microspheres

(76) Inventors: Alexander Andrianov, Belmont, MA (US); Bryan Roberts, Cambridge, MA (US)

Correspondence Address: CARELLA, BYRNE, BAIN, GILFILLAN,, CECCHI, STEWART & OLSTEIN, 5 Becker Farm Road, Roseland, NJ, 07068, US

(21) Appl. No.: 10/715,787

(22) Filed: Nov. 18, 2003

Related U.S. Application Data
(60) Provisional application No. 60/428,310, filed on Nov. 22, 2002.

(51) Int. Cl. A61K 9/16; A61K 9/50; C12Q 1/70; B01J 13/02; B01J 13/04

(52) U.S. Cl. 424/490; 264/4.1

(57) ABSTRACT

Methods of producing polyphosphazene microspheres comprising admixing aqueous solutions of a water-soluble polyphosphazene and an organic amine, or salt thereof, are disclosed.

Polymer microspheres find numerous uses both in the life sciences and in industrial applications. Kawaguchi, H., Functional Polymer Microspheres, Prog. Polym. Sci, Vol. 25, pp. 1171-1210, 2000. Medical and biochemical applications include their use as pharmaceutical carriers for a variety of prophylactic and/or therapeutic agents; their use in biospecific separation, immunoassay and affinity diagnosis; and their use as immunoadjuvants. Microspheres also attract attention as materials for optical, opto-electrical, and rheological applications. Methods for preparation of synthetic polymer microspheres have been described, however these methods are laborious, consist of multiple steps, and require the use of organic solvents, surfactants, and harsh reaction conditions. Examples of such methods are described elsewhere. Polymeric Nanoparticles and Microspheres, Guiot, P. and Couvreur, P., Eds., CRC Press, Inc., Boca Raton, Fla., 216p., 1986.

Polyphosphazene hydrogel micro/nanospheres are of great importance for use in both biomedical and industrial applications because of their biocompatibility, biodegradability, and several other important properties originating from their unusual inorganic backbones. Aqueous based synthetic processes for their preparation attract special attention because of simplicity, safety, and the mild conditions under which they can facilitate encapsulation. Methods for their preparation have been described previously, such as by spraying an aqueous polyphosphazene solution into a solution containing multivalent metal cations. Burgess, D. J. (1994) Complex Coacervation: Microcapsule Formation. In: Dubin, P., Bock, J., Davis, R., Schulz, D. N. and Thies, C. (Eds.), Macromolecular Complexes in Chemistry and Biology, Springer-Verlag, Berlin, Heidelberg, New York, London, Paris, Tokyo, Hong Kong, Barcelona, Budapest, pp. 285-300. The process, however, requires complicated spraying equipment, presents potential safety problems when used to encapsulate potentially hazardous materials, and allows no, or limited, control over the microsphere size distribution. Alternatively, ionically cross-linked polyphosphazene hydrogel microspheres can be prepared by a coacervation in an aqueous solution which requires a two step process comprised of microdroplet formation induced by monovalent cations, and microdroplet stabilization by ionic cross-linking with salts of multivalent metal cations.

It is an object of the present invention to provide a method of producing stable polyphosphazene microspheres. Such microspheres are produced by incubating a solution that contains the polyphosphazene and an organic amine for a period sufficient to produce microspheres.

It is a further object of the present invention to provide a method for encapsulating biological materials by mixing biological material with polyphosphazene solution before microsphere preparation.

The term “coacervation” as used herein means the separation of a macromolecular solution into two immiscible liquid phases. One phase is a dense coacervate phase, concentrated in the macromolecules and forming droplets, and the other phase is a polymer deficient phase. Coacervation is a result of a molecular dehydration of the polymer. Coacervation may be induced by a temperature change, addition of a non-solvent or addition of a micro-salt (simple coacervation), or by the addition of another polymer thereby forming an interpolymer complex (complex coacervation). Coacervates may be described as liquid crystals and mesophases and are more fluid than other systems with higher structural order, such as micelles. Such systems are in dynamic equilibrium and change in the conditions may result in either the reformation of a one phase system or the formation of a flocculate or precipitate. Burgess, D. J. (1994) Complex Coacervation: Microcapsule Formation. In: Dubin, P., Bock, J., Davis, R., Schulz, D. N. and Thies, C. (Eds.), Macromolecular Complexes in Chemistry and Biology, Springer-Verlag, Berlin, Heidelberg, New York, London, Paris, Tokyo, Hong Kong, Barcelona, Budapest, pp. 285-300.

The advantages of the method for making microspheres using coacervation are that it avoids the use of organic solvents, heat, complicated manufacturing equipment (such as spray equipment), and eliminates the generation of aerosol. The method is highly reproducible and generates microspheres with an improved, more narrow microsphere size distribution, compared to the spray technique. Unlike the microspheres obtained by spray methods, coacervation-produced microspheres do not contain a significant amount of larger sized aggregates or amorphous precipitates. This result is important for the preparation of microspheres for vaccine delivery, since the uptake of these microspheres by M-cells is limited to the particles having diameters of 10 μm or less. A further advantage of the coacervation process is that it enables the efficient control of the microsphere size by simply varying the concentration of the components. A particular advantage of the herein-described coacervation by amine method is its ability to form nanospheres—microspheres having diameters of less than 1 micron. Neither the spray methods nor the two step monovalent coacervate/multivalent cross-linking cation methods are effective at producing microspheres of such diminutive size. This aspect of the present invention also results in decreased aggregation, a problem occurring when a small percentage of the total number of microspheres are inordinately voluminous, and as result contain an overwhelming percentage of the materials intended to be encapsulated.

A further advantage of the amine coacervate method over the prior art is that it is essentially a single step process. As the coacervation agent, the amine initiates microdroplet formation through electrostatic screening that decreases the polymer's solubility and causes the polymer to collapse. As the cross-linking agent, the amine decreases the polymer's chain mobility and thereby arrests the growth of the microdroplet at the desired size.

Polyphosphazenes are polymers with backbones consisting of alternating phosphorus and nitrogen atoms, separated by alternating single and double bonds. Each phosphorous atom is covalently bonded to two pendant groups (“R”). The repeated unit in polyphosphazenes has the following general formula:

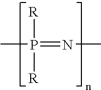

wherein n is an integer.

Phosphorous can be bound to two like groups, or two different groups. In general, when the polyphosphazene has more than one type of pendant group, the groups will vary randomly throughout the polymer, and the polyphosphazene is thus a random copolymer. Polyphosphazene with two or more types of pendant groups can be produced by reacting poly(dichlorophosphazene) with the desired nucleophile or nucleophiles in a desired ratio. The resulting ratio of pendant groups in the polyphosphazene will be determined by a number of factors, including the ratio of starting materials used to produce the polymer, the temperature at which the nucleophilic substitution reaction is carried out, and the solvent system used. While it is difficult to determine the exact substitution pattern of the groups in the resulting polymer, the ratio of groups in the polymer can be easily determined by one skilled in the art.

Phosphazene polyelectrolytes are defined here as polyphosphazenes that contain ionic (ionized or ionizable) pendant groups, which groups impart to the polyphosphazene anionic, cationic, or amphiphilic character. The ionic groups can be in the form of a salt, or, alternatively, an acid or base that is, or can be, at least partially dissociated. Any pharmaceutically acceptable monovalent cation can be used as counterion of the salt, including but not limited to sodium, potassium, and ammonium. The phosphazene polyelectrolytes can be biodegradable or non-biodegradable under the conditions of use.

A preferred phosphazene polyelectrolyte is a polyanion and contains pendant groups that include carboxylic acid, sulfonic acid, hydroxyl, or phosphate moieties. While the acidic groups are usually on non-hydrolysable pendant groups, they can alternatively; or in combination, also be positioned on hydrolysable groups. An example of a phosphazene polyelectrolyte having carboxylic acid groups as side chains is shown in the following formula:

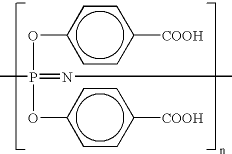

wherein n is an integer, preferably an integer between 10 and 300,000, and preferably between 10,000 to 300,000. This polymer has the chemical name poly[di(carboxylatophenoxy)phosphazene] or, alternatively, poly[bis(carboxylatophenoxy) phosphazene], (PCPP).

The phosphazene polyelectrolyte is preferably biodegradable to prevent eventual deposition and accumulation of polymer molecules at distant sites in the body, such as the spleen. The term biodegradable, as used herein, means a polymer that degrades within a period that is acceptable in the desired application, typically less than five years and most preferably less than about one year, once exposed to a physiological solution of pH 6-8 at a temperature of approximately 25° C.-37° C.

Polyphosphazenes, including phosphazene polyelectrolytes, can be prepared by a macromolecular nucleophilic substitution reaction of poly(dichlorophosphazene) with a wide range of chemical reagents or mixture of reagents in accordance with methods known to those skilled in the art. Preferably, the phosphazene polyelectrolytes are made by reacting the poly(dichlorophospahzene) with an appropriate nucleophile or nucleophiles that displace chlorine. Desired proportions of hydrolyzable to non-hydrolyzable side groups or ionic to non-ionic side groups in the polymer can be obtained by adjusting the quantity of the corresponding nucleophiles that are reacted with poly(dichlorophosphazene) and the reaction conditions as necessary. Preferred polyphosphazenes have a molecular weight of over 1,000 g/mol, most preferred between 500,000 and 1,500,000 g/mol.

The polyphosphazene may be contained in an appropriate solution, such as, for example, water, phosphate buffered saline (PBS), inorganic or organic buffer solutions, aqueous solutions of biological materials, proteins, antigens, or mixtures thereof. The polyphosphazene may be present in the solution at any concentration, pH, or ionic strength, preferably in concentrations from about 0.01% to about 1.5%, and between pH 7 and pH 8.

In the present invention the polyphosphazene solution is admixed with a solution containing at least one organic amine, or a salt thereof. In one embodiment, the organic amine is spermine or spermidine. The organic amine may be present in the solution at any concentration and pH, preferably from about 0.01% to about 40%, and a pH between 7 and 8. The amine is preferably a water-soluble amine.

The resulting mixture containing polyphosphazene and the organic amine solution is allowed to stand for a period of time, which is sufficient to allow for the formation of a coacervate phase; i.e., coacervate microdroplets of polyphosphazene are formed in the mixture. Alternatively, the organic amine is fed to the reaction mixture over an extended period of time. In yet another embodiment, both the polyphosphazene solution and the organic amine solution are fed to the reaction mixture over an extended period of time. The kinetics of microsphere formation and growth can be followed by observing the mixture with an optical microscope or by measuring the particle size distribution with a particle size analyzer. The reaction mixture can be agitated by stirring, vortexing, or shaking, or it can be allowed to stand without agitation. The coacervate microspheres can be stabilized at any time. For example, once the desired parameters, of size and size distribution are reached, the microspheres can be stabilized by a simple dilution of the reaction mixture with water or aqueous buffer solution. Aqueous buffer solutions of variable pH and ionic strength can be used, most preferably aqueous buffer solutions with pH between 4 and 7 are used. Alternatively, the coacervation mixture can be allowed to stand until an equilibrium between the coacervate phase and the solution is reached. The microspheres may then be recovered from the suspension by methods known to those skilled in the art, such as, for example, by centrifugation, filtration, or freeze-drying. A further advantage of the herein-described coacervation by amine method, is its ability to form microspheres that are exceptionally stable under physiological conditions; those microspheres ionically crosslinked by multivalent Calcium, in the two step prior art method, are not stable at a pH of 7.4 in the presence of monovalentions.

In general, where materials are to be encapsulatated, the materials are mixed with the polyphosphazene solution prior to coacervation to insure dispersion of the antigen throughout the microsphere. In another embodiment, the material to be encapsulated is fed to the reaction mixture over an extended period of time.

In another embodiment, the microspheres are formed by preparing a water-soluble, inter-polymer complex comprising a polyphosphazene and another water-soluble polymer capable of forming such complex through electrostatic, hydrogen, or hydrophobic interactions. In one embodiment such a polymer is a polyelectrolyte. In yet another embodiment such water-soluble polymer is one that is capable of hydrogen bonding. The inter-polymer complex can be formed at any molecular ratios except those that cause precipitation. The complex can also be formed at any pH, ionic strength, or temperature, but pH ranges from 7 to 8 are preferred, as are conditions of room temperature. Induction of coacervation then, is effected by the addition of a solution of an organic amine, such as hereinabove described to form inter-polymer complex coacervate microspheres.

The preparation of polyphosphazene microspheres by coacervation enables one to recover an increased yield of polyphosphazene microspheres having a size in the micron range (up to 90 differential percent by volume and 95 differential percent by number) and to produce microspheres of other sizes, without the use of elaborate equipment.

The microspheres, formed by coacervation, as herein-described, may be employed as carriers for a variety of prophylactic or therapeutic agents. In one embodiment, the microspheres may be employed as carriers of an antigen capable of eliciting an immune response in an animal. The antigen may be derived from a cell, bacterium, virus particle, or any portion thereof. The antigen may be a protein, a peptide, a polysaccharide, a glycoprotein, a glycolipid, a nucleic acid, or any combination thereof that elicits an immune response in an animal, including mammals, birds, and fish. The immune response may be a humoral immune response or a cell-mediated immune response. Where the material against which an immune response is directed is poorly antigenic, such material may be conjugated to a carrier such as albumin, or to a hapten, using standard covalent binding techniques. Such conjugation can be effected with commercially available reagent kits that are well known in the art.

In one embodiment, the microspheres are employed to deliver a nucleic acid sequence that encodes an antigen to a mucosal surface where the nucleic acid is expressed.

As non-limiting examples of antigens that may be contained in the polyphosphazene microspheres there may be mentioned viral proteins, such as influenza proteins, human immunodeficiency virus (HIV) proteins, Herpes virus proteins, and hepatitus A and B proteins. Additional examples include antigens derived from rotavirus, measeles, mumps, rubella, and polio; or from bacterial proteins and lipopolysaccharides such as Gram-negative bacterial cell walls. Further antigens may also be those derived from organisms such as Haemophilus influenza, Clostridium tetani, Corynebacterium diphtheria, and Nesisseria gonhorrhoae.

The antigen-containing microspheres can be administered as a vaccine by any method known to elicit an immune response. Such methods can be parenteral, or by trans-membrane or trans-mucosal administrations. Preferably, the vaccine is administered parenterally (intravenously, intramusculary, subcutaneously, intraperitoneally, etc.), and subcutaneously. Non-limiting examples of routes of delivery to mucosal surfaces are intranasal (or generally, the nasal associated lymphoid tissue), respiratory, vaginal, oral, and rectal.

The dosage is determined by the antigen loading and by standard techniques for determining dosage and schedules for administration for each antigen, based on titer of antibody elicited by the microspheres antigen administration.

The encapsulated material may also be any other biologically active synthetic compound.

### BRIEF DESCRIPTION OF THE DRAWINGS

The invention now will be described with respect to the drawings, wherein:

FIG. 1 is a phase diagram for a coacervation system formed by mixing aqueous solutions of PCPP and spermine;

FIG. 2 is a graph of the differential percentages of microspheres by number (1) and by volume (2) for microspheres prepared with 0.19% PCPP and 7% spermine in phosphate buffered saline (pH 7.4, 60 min.);

FIG. 3 is a graph of the differential percentages of microspheres by number for microspheres prepared with 0.19% (1) and 0.38% (2) of PCPP (pH 7.4, 7% spermine, 60 min.).

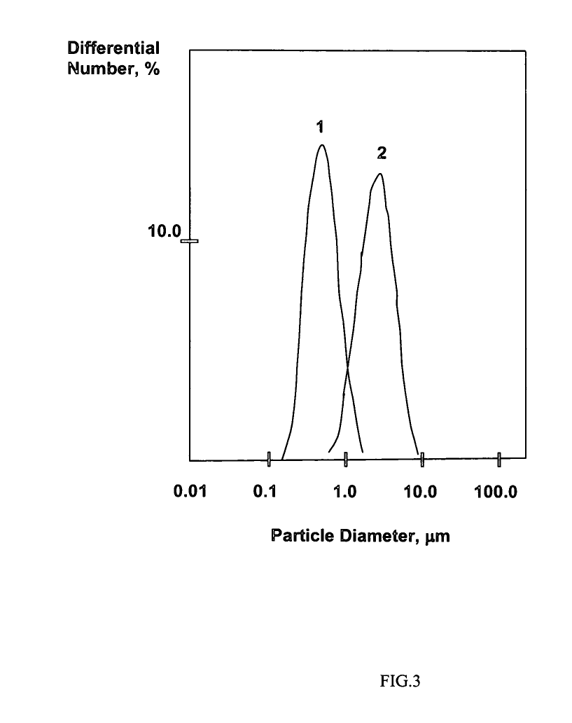

[image]

[image]

[image]

### DETAILED DESCRIPTION OF THE INVENTION

The invention is further described below by several illustrative examples. These examples are added to the preceding instruction for the sole purpose of further enabling the artisan of ordinary skill to make and practice the applicants' best mode of the invention. They are not intended to limit the scope of the claims appended hereto.

### EXAMPLE 1

### Polyphosphazene—Organic Amine Coacervate Systems

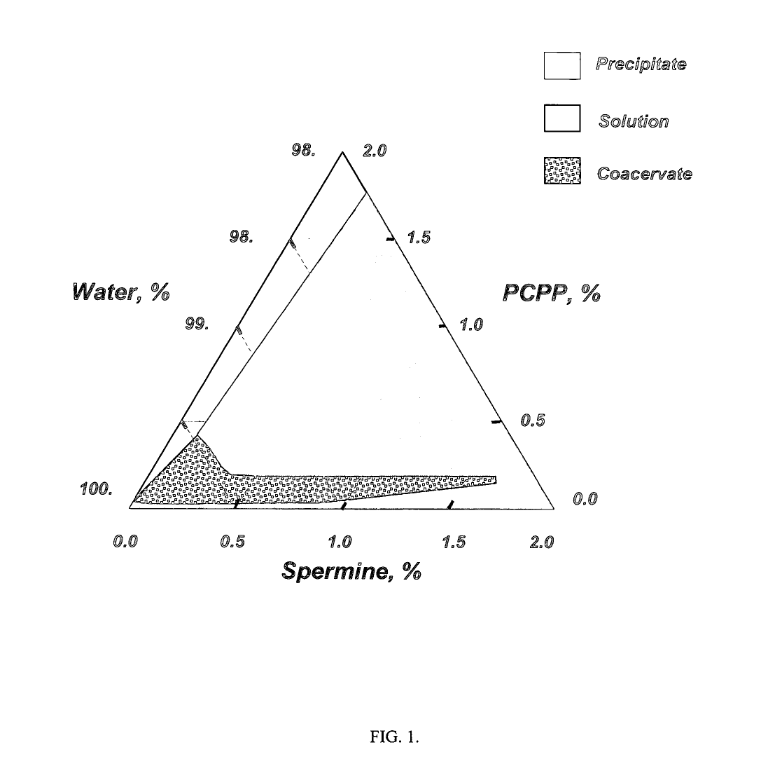

The ability of polyphosphazenes to form coacervate systems in the presence of organic amines was demonstrated using aqueous solutions of PCPP and spermine tetrahydrochloride. A phase diagram of a polyphosphazene—spermine—water system was prepared as follows. Sodium salt of PCPP (weight average molecular weight 8.4×10⁵ g/mol) was dissolved in deionized water to prepare a series of solutions ranging in concentration from 0.002 to 3.6% (w/v). Solutions of spermine in deionized water were prepared ranging in concentration from 0.02 to 12% (w/v). The polymer solutions were then mixed with the spermine solutions in the ratio of 1.0 ml to 0.2 ml, so that the concentration of PCPP and spermine in the resulting solutions varied in the 0 to 2% (w/v) range. The solutions or dispersions were agitated by gentle shaking and then examined by microscope to determine the presence of coacervate droplets or precipitate. The phase diagram was then established by plotting the physical state of the system versus composition of the tertiary system—spermine, PCPP, and water (FIG. 1). The diagram contains three regions—coacervate, precipitate, and homogeneous solution.

### EXAMPLE 2

### Preparation of PCPP—Spermine Hydrogel Microspheres

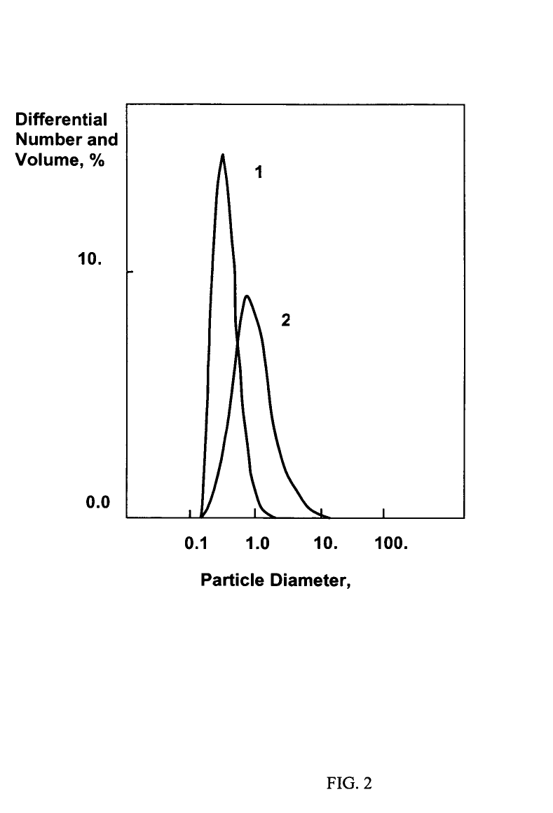

PCPP microspheres were prepared in a single step coacervation process using the physiologically acceptable organic amine, spermine, as both the coacervating and the cross-linking agent. 0.07 ml of 7% solution of spermine in PBS (pH 7.4) were added to 5 ml of 0.19% aqueous PCPP solution (PBS, pH 7.4) and were agitated gently by shaking. The mixture was then incubated at ambient temperature for 60 minutes. The suspension of microspheres was then diluted with a three-fold excess of PBS buffer (pH 6.5), was let to stand for additional 30 minutes, and was thereafter examined for the presence of particulates using a Mastersizer S (Malvern instrument Ltd.). FIG. 2 shows differential percentages of microspheres by number (1) and by volume (2) demonstrating narrow particle size distribution. The mean diameters were 0.41 μm and 1.52 μm by number and by volume respectively.

### EXAMPLE 3

### Preparation of PCPP—Spermine Microspheres of Variable Size

The effect of polymer and spermine concentration on microsphere size was investigated. 0.19% (w/v) and 0.38% (w/v) aqueous PCPP solutions (PBS buffer, pH 7.4) were prepared in the amount of 3.8 ml of each. To these solutions 0.04 ml and 0.08 ml of 7% (w/v) spermine solution in PBS (7.4) were added respectively, so that the molar concentration of PCPP to spermine was kept the same for both mixtures (3.5:1). The mixtures were then incubated at ambient temperature for 60 minutes and particle size distribution of the resulting microspheres was analyzed using a Mastersizer S (Malvern instrument Ltd.). The results demonstrated the formation of particulates with a sub-micron size for the lower PCPP—spermine concentration (0.51 μm by volume) and larger microspheres (1.79 μm by volume) for a mixture with higher concentration. Thus, varying total PCPP:spermine concentration in the reaction mixture allows for an effective control of microsphere—nanosphere size distribution.

We claim:

1. A method of producing polyphosphazene microspheres comprising:
(a) admixing an aqueous solution containing a water-soluble polyphosphazene and an aqueous solution containing an organic amine, or a salt thereof, and
(b) allowing the reaction mixture to stand for an effective period of time to form thereby polyphosphazene microspheres.

2. The method of claim 1, wherein said water-soluble polyphosphazene and said organic amines are fed to the mixture over an extended period of time.

3. The method of claim 1, further comprising adding water or aqueous buffer solution to stabilize the microspheres.

4. The method of claim 1, further comprising recovering said polyphosphazene microspheres.

5. The method of claim 1 wherein said organic amine is spermine.

6. The method of claim 1 wherein said polyphosphazene is poly[di(carboxylatophenoxy)phosphazene].

7. The method of claim 1 wherein said microspheres have diameters of from about 1 μm to about 10 μm.

8. A method of producing polyphosphazene microspheres containing material to be encapsulated comprising:
(a) admixing an aqueous solution containing a water-soluble polyphosphazene and an aqueous solution containing material to be encapsulated to form a reaction mixture;
(b) then admixing to said reaction mixture an aqueous solution containing an organic amine, or a salt thereof;
(c) allowing the reaction mixture to stand for an effective period of time to form thereby polyphosphazene microspheres;

9. The method of claim 8 wherein said material is a biologically active material selected from the group consisting of proteins, biologically active synthetic compounds, nucleic acids, polysaccharides, and antigens.

10. The method of claim 9 wherein said antigen is derived from organisms selected from the group consisting of rotovirus, measles, mumps, rubella, polio, hepatitis A, hepatitis B, herpes virus, human immunodeficiency virus, influenza virus, Haemophilus influenza, Clostridium tetani, Corynebacterium diphteria, and Neisseria gonorrhea.

11. A vaccine comprising the polyphosphazene microspheres made by the methods of claims 8, 9, or 10.
